(19) United States
(12) Patent Application Publication
(10) Pub. No.: US 2016/0038018 A1
(43) Pub. Date: Feb. 11, 2016

(54) MERGED TROCAR-OBTURATOR DEVICE FOR OPTICAL-ENTRY IN MINIMALLY INVASIVE SURGERY

(71) Applicant: VANTAGE SURGICAL SYSTEMS, INC., Irvine, CA (US)

(72) Inventors: Jason Wilson, Los Angeles, CA (US); Vacit Arat, La Canada Flintridge, CA (US)

(21) Appl. No.: 14/774,863

(22) PCT Filed: Mar. 11, 2014
(86) PCT No.: PCT/US14/23152

§ 371 Date: Sep. 11, 2015

Related U.S. Application Data
(60) Provisional application No. 61/780,281, filed on Mar. 13, 2013.

(51) Int. Cl. A61B 1/313; A61B 17/34; A61B 1/015; A61B 1/12; A61B 1/00; A61B 1/06

(52) U.S. Cl. CPC A61B 1/3132; A61B 1/00154; A61B 1/00085; A61B 1/00096; A61B 1/0676; A61B 1/015; A61B 1/126; A61B 17/3423; A61B 2017/3484

(57) ABSTRACT

The present invention provide for improved optical entry systems and methods for minimally invasive surgery. According to some aspects of the disclosure, an obturator and trocar are merged to provide a device that can be used with an integrated visualization means (e.g. laparoscope) to provide optical entry into a patient's body cavity. Further, a reconfigurable tip is configured to be in an entry state and a visualization state. Said aspects and associated method steps can significantly reduce the complexity of the entry process and do not require removal and reinsertion of surgical instruments which eliminates the need for valves in the trocar and reduces the possibility of contamination of the visualization means' objective lens.

### RELATED APPLICATIONS

This application is a non-provisional application of U.S. Provisional Application No. 61/780,281, filed on Mar. 13, 2013.

### FIELD OF THE INVENTION

The present invention relates generally to the field of minimally invasive surgical instruments involving optical visualization systems. More specifically, the minimally invasive surgical instruments and procedures comprising trocars and/or cannulas that can be used to create a minimally invasive punctures into the body and which may retain optical properties of the optical components.

### BACKGROUND OF THE INVENTION

Minimally invasive surgery (MIS) is the technique of performing surgery through small incisions (less than two centimeters) or punctures in the body. During these surgical procedures visualization can be achieved using optical devices such as endoscopes, laparoscopes, arthroscopes, boroscopes and the like; this is contrasted to open surgery performed through large incisions with direct visualization. Two surgical instruments typically used to introduce the visualization device into the body is a trocar and obturator. The obturator can be used to create the incision or puncture through tissue and the trocar to maintain tissue or an opening in an open position. Once the trocar is in place, the visualization device can be inserted in to the body cavity. In some embodiments, trocars can also be used to facilitate the introduction of other surgical instruments as well as other functions such as providing a means for gases to pass through for insufflation purposes.

Typically the trocar can fit over the obturator during insertion. In the case of laparoscopic surgery, the surgeon can push the trocar and obturator through the abdominal wall until the distal end of the trocar has been introduced into the body cavity and is proximal to the surgery site. The obturator is then removed while the trocar holds the incision open, allowing the laparoscope to be introduced in to the abdominal cavity.

A preferred procedure for entry using a trocar and obturator is known as visual entry. In the visual entry procedure, the tip of the obturator is transparent, and a laparoscope is placed inside the obturator during entry. From this location the laparoscope may be used to image the tissue visible through the transparent obturator tip. The standard visual entry procedure includes the following six steps: 1) The obturator is inserted into the trocar; 2) The endoscope is inserted into the obturator; 3) The three instruments are pushed through the abdominal wall by the surgeon who may simultaneously monitors the progress on a video screen; 4) The laparoscope is then removed; 5) The obturator is then removed; and finally, 6) the endoscope can be reinserted for visualization during surgery.

This standard procedure for visual entry is cumbersome as it requires many steps to remove and reinsert various surgical instruments as described above. For example, valve systems are required to seal the surgical site during removal and reinsertion. Furthermore, biological contaminants can be introduced into the trocar during removal of the obturator which can contaminate the lens of the laparoscope upon reinsertion impairing the vision of the surgeon or exposing the surgical site to biological contaminants.

It is desirable therefore to have novel devices, systems, and/or tools that can facilitate procedures by eliminating one or more of the steps, and thereby various associated risks, that occur during surgical procedures.

### SUMMARY OF THE INVENTION

The foregoing needs are met, to a great extent, by the present invention, in which a system for use in minimally invasive surgery for introducing a visualization device into a body cavity is disclosed.

According to some aspects of the disclosure, a system for use in minimally invasive surgery for introducing a visualization device into a body cavity is provided. The system can include a tube, a visualization device disposed inside the tube; and a distal tip configured of being in either an entry state or a visualization state and adapted for the tube. The entry state can be characterized by a rigid tip geometry capable of puncturing through a body wall into an anatomical cavity and the visualization state by a tip geometry that has substantially articulated away from an optical axis of the visualization device disposed in the tube.

In some embodiments, the tube can include auxiliary channels and/or integrated lighting means. The auxiliary channels may be used for the introduction of gases or liquids during one or more of the visualization steps and/or for the surgery. For example, the gases may be used for insufflation of a surgical area. The liquids may be active agents and/or liquids used to rinse clean an objective lens of the visualization device.

In additional aspects of the disclosure, a method for use of a visualization device in minimally invasive surgery is disclosed. The method can include: (1) puncturing a body wall with a transparent reconfigurable arranged with a tube to include a visualization device, wherein the transparent reconfigurable tip includes a distal end and a proximal end and the distal end is configured in an entry state during the puncturing; (2) monitoring the puncturing progress as imaged by the visualization device; (3) actuating the reconfigurable tip to transition from the entry state to a visualization state once the reconfigurable tip is the body cavity; and (4) adjusting the visualization device to image a surgical site. In some embodiments the method can additionally include: actuating an integrated lighting means contained in the transparent reconfigurable tip during one or more of the puncturing step, the actuating of the reconfigurable tip, and the adjusting of the visualization device; and/or the introduction of a liquid or a gas through one or more auxiliary channels of the tube. Further, the steps of monitoring and adjusting may be performed without having to replace or temporarily remove the imaging device arranged inside of the tube. Similarly, the steps of puncturing, monitoring, actuating, and adjusting can be performed without using valve systems for the removal and reinserting of additional surgical instruments utilized for the viewing of the surgical site.

There has thus been outlined, rather broadly, certain aspects of the invention in order that the detailed description thereof herein may be better understood, and in order that the present contribution to the art may be better appreciated. There are, of course, additional aspects of the invention that will be described below and which will also form the subject matter of the claims appended hereto.

In this respect, before explaining at least one aspects of the invention in detail, it is to be understood that the invention is not limited in its application to the details of construction and to the arrangements of the components set forth in the following description or illustrated in the drawings. The invention is capable of aspects in addition to those described and of being practiced and carried out in various ways. Also, it is to be understood that the phraseology and terminology employed herein, as well as the abstract, are for the purpose of description and should not be regarded as limiting.

As such those skilled in the art will appreciate that the conception upon which this disclosure is based may readily be utilized as a basis for the designing of other structures, methods and systems for carrying out the several purposes of the invention. It is important, therefore, that the claims be regarded as including such equivalent constructions insofar as they do not depart from the spirit and scope of the invention.

### BRIEF DESCRIPTION OF THE DRAWINGS

The above mentioned features and aspects of the disclosure will become more apparent with reference to the following description taken in conjunction with the accompanying drawings wherein like reference numerals denote like elements and in which:

FIG. 1a is a side view of an exemplary embodiment of the merged trocar-obturator device in the entry state according to various aspects of the present disclosure;

FIG. 1b is a side view of the merged trocar-obturator device of FIG. 1a in the visualization state according to various aspects of the present disclosure;

FIG. 2a is a cross section of an exemplary embodiment of a reconfigurable tip in the entry state;

FIG. 2b is a cross section of the exemplary embodiment of FIG. 2a with the reconfigurable tip in the visualization state according to various aspects of the present disclosure;

FIG. 3 is an isometric view of the reconfigurable tip in the entry state according to various aspects of the present disclosure;

FIG. 4 is a view of the proximal end of a reconfigurable tip of FIG. 3 in the entry state according to various aspects of the present disclosure;

FIG. 5 is an isometric view of an alternative exemplary embodiment of the reconfigurable tip in the visualization state with integrated illumination means according to various aspects of the present disclosure;

FIG. 6 is a side view of the exemplary merged trocar-obturator device of FIG. 5 disposed through a body wall and introducing the visualization device into the body cavity according to various aspects of the present disclosure; and

FIG. 7 is a flowchart with exemplary method steps that may be used according to aspects of the systems of the present disclosure.

### DETAILED DESCRIPTION OF PREFERRED EMBODIMENTS

The present disclosure provides for a system in which, in some embodiments, one or more of these problems can be addressed by the merging of the obturator and the trocar into a single surgical device. According to some aspects, this can be accomplished by a reconfigurable tip, preferably transparent, with at least two states—an entry state and visualization state. In the entry state the tip can be closed and/or sealed, and form a tip geometry that can allow the instrument to puncture patient's tissue, e.g., the patient's outer tissue. In this configuration, the image capturing end of a visualization device can be placed proximal to or touching the closed and/or sealed tip.

In the visualization state, the tip can be substantially displaced or articulated into the body cavity to provide a relatively unobstructed view of the surgical field as it may be imaged by the visualization device. In one embodiment, for example, the tip can include at least one transparent component that allows light to enter the visualization device allowing tissue to be imaged through the tip providing visual entry.

The tip may be able to substantially rotate about the approximately outside diameter of the trocar as to not interfere with the optical path of the visualization device and providing an unobstructed view of the surgical site, for example. This rotation can be facilitated by a standard hinge, a compliant flexure, or the like. The tip may be an integral component of the trocar, attached, or a separate component that is movably attached to the trocar.

According to additional aspects, one or more tips may be included. One or more of the tips, each preferably being transparent, may include a plurality of partial tips with independent hinges. When in the entry state, the various tips can connect to create a substantially rigid tip with a geometry capable of piercing an abdominal wall, i.e., a sharp or pointed structure. When in the visualization state, the partial tip components can disengage and articulate away from the visualization device independently. In some embodiments, the transition from entry state to visualization state can be achieved by actuating the tip using the visualization device itself. For example, using forces from the dissection which can tend to keep the tip in the entry state, while forces from the visualization device can push on the proximal end of the tip causing a transition to the visualization state.

In this specification and claims it is to be understood that reference to a “trocar” or “trocar device” is intended to encompass a cannula or tube that may be inserted through an incision or tissue to maintain it open, for example, to allow surgical instruments and/or visualization devices to be inserted into a body cavity.

The term “obturator” or “obturator device” is intended to encompass any device that can be placed into, distally attached or integrated, into a trocar to prevent the trocar from being blocked by any tissue during insertion. Furthermore, an obturator can include a tip or sharp geometry that substantially dissects the tissue and can allow for easier insertion of the trocar. By definition, the obturator must be removed or substantially disposed away from the trocar to allow for a considerably/completely unobstructed view of the surgical area. The obstructed view means any occlusion or aberration of light prior to image capture by the visualization device.

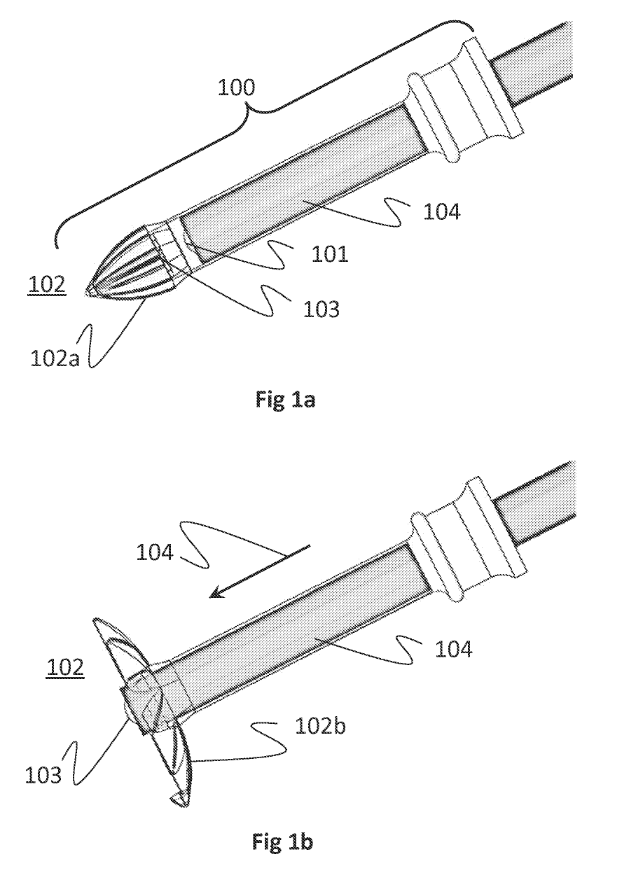

Referring now to FIG. 1a and FIG. 1b, side views of an exemplary embodiment of a merged trocar-obturator device 100 are depicted. In particular, FIG. 1a depicts the exemplary merged trocar-obturator device 100 with a reconfigurable tip 102 in the entry state 102a. Regardless of the reconfigurable geometry of the reconfigurable tip 102, the entry state 102a can be characterized by a rigid tip geometry capable of dissecting or puncturing tissue. This geometry can be the result of a single movable tip (e.g. reconfigurable tip 102), or in additional embodiments, a rigid structural interaction between or combination of multiple tip components. This reconfigurable tip 102 geometry during the entry state 102a could be a sharp point, blade, or cutter as well as blunt. By blunt it is meant that small force interaction with human tissue does not cause cutting or dissection; there is also some force threshold above which interaction with human tissue does cause cutting or dissection.

Disposed inside of the trocar 104 can be a visualization device 101, as depicted in FIG. 1a when the reconfigurable tip 102 is in the entry state 102a. The visualization device 101 can be an endoscope, laparoscope, arthroscope, borescope or the like. In the entry state 102a, the distal end of the visualization device 101 can be directed at the back portion of the reconfigurable tip 102. In some embodiments, the reconfigurable tip 102 can be clear allowing the visualization device 101 to image tissue (not shown) during insertion, allowing for visual entry.

In particular, FIG. 1b depicts the exemplary merged trocar-obturator device 100 with the reconfigurable tip 102 in the visualization state 102b. According to some aspects, the reconfigurable tip 102 can include a plurality of components that are configured to provide an unobstructed view of the surgical area in the visualization state 102b. In some embodiments, the reconfigurable tip 102 may be disposed such that the distal end of the visualization device 103 can move axially in the direction denoted by 104 such that it enters the body cavity for wide angle visualization that is unobstructed by the trocar 104 or reconfigurable tip 102 during the visualization state 102b.

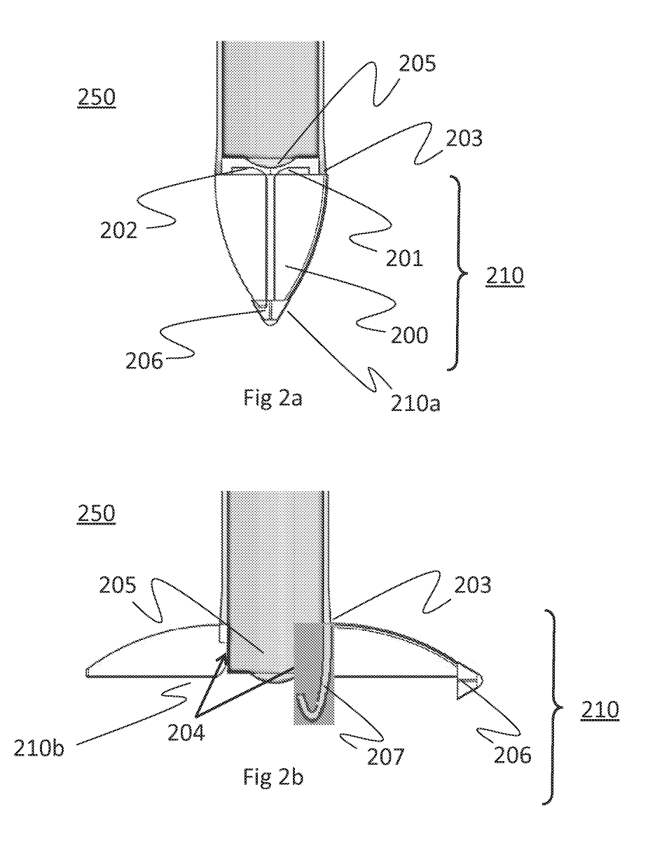

Referring now to FIGS. 2a and 2b, cross sections of an exemplary combined trocar/obturator device 250 including an at least two piece reconfigurable tip 200 are shown. In particular, FIG. 2a is a cross section representation of an exemplary reconfigurable tip 210 at an entry state 210a. In the entry state 210a the at least two components of the reconfigurable tip 210 may be substantially connected by a cap 206 attached to one of the at least two tip components 200 forming a blunt or sharp point that can be suitable for tissue dissection. The distal end of the visualization device 205 can be positioned behind the reconfigurable tip 210. Since the reconfigurable tip 210 can generally be clear/transparent it can be possible for the visualization device 205 to image objects and tissue that is beyond the reconfigurable tip 210 before, during and after insertion into a body cavity.

According to aspects of the present disclosure, after insertion the reconfigurable tip 210 can be articulated by moving the distal end of the visualization system 205 towards the distal end of the reconfigurable tip 210, triggering an actuation means that can cause the tip to articulate out of the way about hinge 203. The actuation could be from the distal end of the visualization system 205 by physically pushing on the proximal surfaces of the tip 201, 202; actuated automatically via spring loading; or by any other exogenous force such as other electromagnetic, pneumatic, hydraulic, cable, pushrod actuation and the like. Hinge 203 could be a standard hinge, a compliant flexure, a flexible tether, a compliant wire or the like. The component of the at least two component reconfigurable tip 210 which the cap 206 can be attached to could be actuated first to free the other components to articulate freely. The cap 206 may serve at least two purposes including substantially holding the components of the reconfigurable tip in the entry state and maintaining the correct tip geometry for entry. The reconfigurable tip 210 may be readily changed from entry state 210a, depicted in FIG. 2a, to visualization state 210b, depicted in FIG. 2b, by articulating the tip component attached to the cap 206 first to free the remaining tip components.

Accordingly, in some embodiments the distal end of the visualization device 205 may actuate the reconfigurable tip 210 so as to move it from the entry state 210a to the visualization state 210b. Component(s) attached to the cap 206 can be actuated first by extending the proximal surface 201 further than the proximal surfaces of the component(s) with no cap 202 such that the distal end of the visualization system comes in contact with proximal surface 201 before proximal surface 202. After the reconfigurable tip 210 is configured in the visualization state 210b, the reconfigurable tip 210 can be maintained as such by physical interference between the visualization device 205 and the tip's proximal surfaces 201 and 202 as shown by 204.

In some embodiments, an auxiliary channel 207 may be integrated in the reconfigurable tip 210 of the trocar/obturator device 250. The auxiliary channel 207 may be curved to direct liquid or gas at the distal end of the visualization device 205, the purpose of cleaning the objective lens from debris that may have contaminated it, for defogging purposes, and/or for administration of an active agent such as an analgesic.

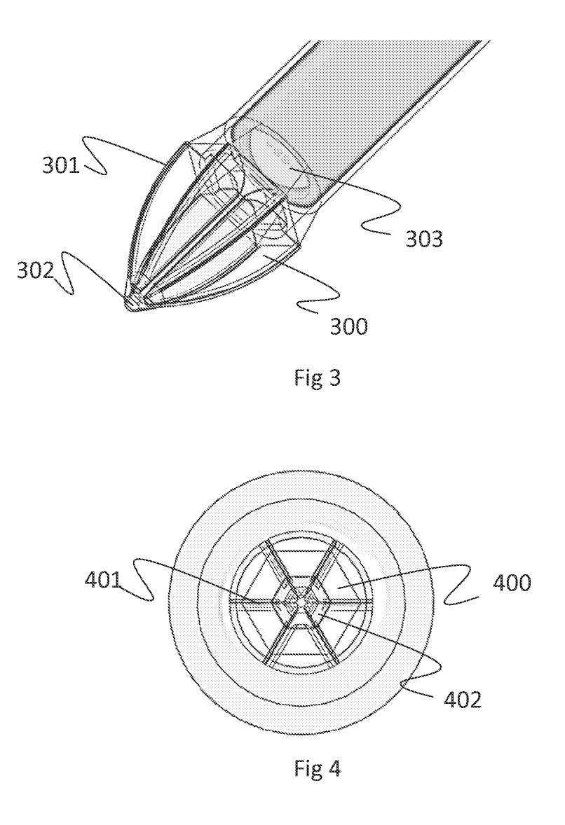

Referring now to FIG. 3, an isometric view of the reconfigurable tip in the entry state is depicted. In particular, the isometric view shows the reconfigurable tip 300 being transparent so as to allow the visualization device 303 to image body wall tissue during entry into a body cavity using the cap 302 having a blunt geometry for entry through body wall tissue into a body cavity. To seal the mating surfaces of a plurality of tip components, a flange 301 substantially attached to one tip component or integrally part of the component's structure may extend to partially cover an adjacent component sealing the interface from tissue introduction. The flange structure 301 may also provide beneficial surface geometry to further enhance tissue dissection or puncture during entry. Accordingly, depending on the flange structure, it may be necessary to actuate the plurality of tip components in sequence to transition from the entry state to the visualization state avoiding binding of components.

Referring now to FIG. 4, a view of the proximal end of the reconfigurable tip as seen through the trocar or tube is depicted. The transparent spaces 400 denote areas of the reconfigurable tip where light can be imaged by the visualization device without being obstructed by reconfigurable the tip geometry. As previously mentioned, the sealing flanges 401 may extend from one component to cover an adjacent component to seal the interfacing surfaces from tissue introduction during entry. The proximal geometry of the cap 402 may readily cover the adjacent components maintaining the entry state during entry.

Referring now to FIG. 5, an isometric view of an alternative exemplary embodiment of the reconfigurable tip with integrated illumination means is depicted. In particular, the reconfigurable tip 550 can include, for example, six tip components 500. The reconfigurable tip could also include one, two, three or any plurality of tip components depending on design aspects, such as overall size, precision needed and the like. According to aspects of the disclosure, illumination components 501 can be integrated into the reconfigurable tip 550, for example, into the tip components 500 forming part of the reconfigurable tip 550. The illumination components 501 may include, for example, light emitting diodes (LED). Other illumination components 501 may alternatively or additionally include optical fibers in the walls of the trocar or the trocar being one large optical fiber, UV light sources, lasers, phototherapy light sources, and the like. As shown the illumination can be directed parallel to the optical axis of the visualization device 502 as denoted by 504. However, some illumination components may be oriented perpendicular to 504 as denoted by 505. These perpendicularly oriented illumination components may function to illuminate, in the entry state, the tissue during entry. Alternately, the remaining illumination components may be used to illuminate, in the visualization state, the surgical area.

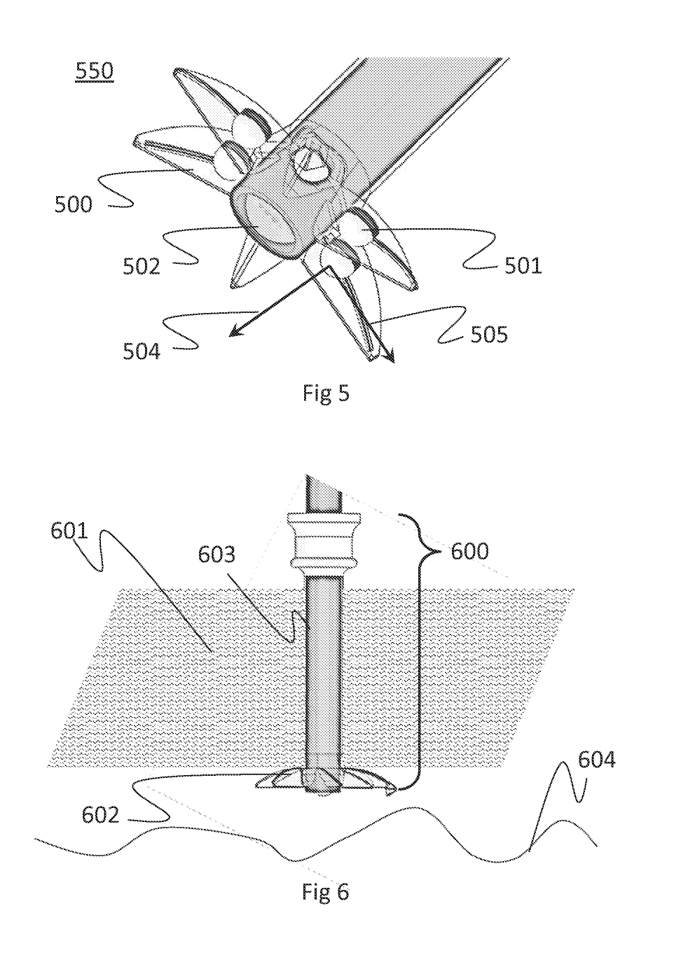

Referring now to FIG. 6, a side view of the exemplary merged trocar-obturator device of FIG. 5 disposed through a body wall is shown. In particular, the trocar/obturator device 600 and visualization device 603 being disposed through a patient's tissue 601 with the reconfigurable tip 602 being in the visualization state. Accordingly, the components of the reconfigurable tip 602 can be articulated away from the visualization device 603 which can offer beneficial purposes beyond providing an unobstructed view of the surgical site 604. For example, the articulated components may be used and designed to hold tissue away from the visualization device 603 as well as to prevent the assembly 600 from being expelled from the incision or puncture. This expulsion from the incision or puncture could happen due to pressure from insufflation, mechanical pulling from the proximal end of the trocar and the like.

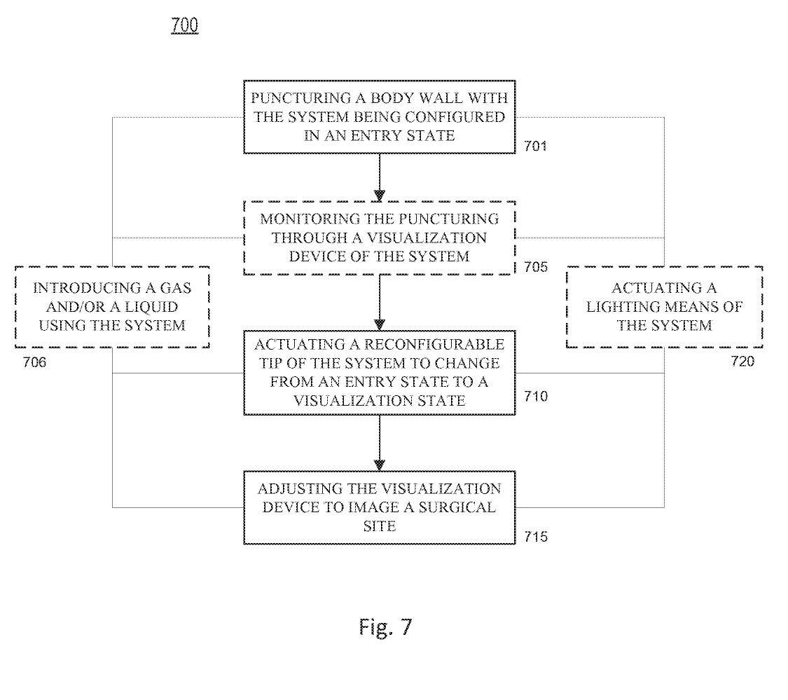

Referring now to FIG. 7, a flowchart with exemplary method steps that may be used according to aspects of the present disclosure is shown. In particular, the exemplary method steps are associated with various embodiments and can be used to simplify the insertion procedure by minimizing risk, provide additional visualization capabilities for improved control by the practitioner, and/or reducing the number of steps that are needed. Before providing further description regarding the steps that may take place, it must be understood that the order of steps may vary, additional steps may take place in between steps, and the order of the exemplary steps presented may occur more than once throughout different stages or omitted depending on the procedure and as it will be apparent to those skilled in the art from the contents of the present disclosure.

At step 701, puncturing of a body wall/tissue can occur. According to aspects of the disclosure, puncturing of the body wall can be done with a transparent reconfigurable tip being at an entry state and arranged with a tube to include a visualization device. At step 705, the puncturing progress may be monitored using the visualization device to image the tissue being punctured. Step 706 may occur during or after the puncturing step 701. In particular, step 706 includes the introduction of one or more liquids and/or one or more gases through at least one auxiliary channel of the tube. Liquid(s) can include, for example, an active agent such as an analgesic, an antibiotic, an enzyme, a defogger, or a non-active agent used to rinse the tip, tissue, the illumination source, and/or the visualization device. One or more gases may also be introduced, for example, to assist in the displacement of debris arising from surgical activity, for insufflation purposes, and the like. For example, gas such as carbon dioxide can flow out of the trocar to aerate the lens of the visualization device to thereby prevent debris/smoke, from the pulverization of tissue that may take place during a surgery, from blocking the field of view of the imaging device.

At step 710, actuation of the reconfigurable tip to transition from the entry state to a visualization state once the reconfigurable tip is the body cavity can occur. This may be done for both visualization purposes or to use the tip components to lock the device onto the tissue. According to some aspects, the visualization device does not need to be removed from the trocar, eliminating the need for valves and sealing means in the proximal end. However, fittings such as luer locks may still be included in the proximal end to introduce liquids or gasses to the auxiliary channels when step 701 takes place. At step 715, the visualization device may be adjusted to image the surgical site. Adjustment may include, for example, adjusting zoom magnification, moving the trocar device itself, focusing an image or changing contrast of an image, and the such. In some embodiments, actuation one or more integrated illuminating means at step 720 may also take place before, during, or after any of the aforementioned steps.

In view of the teachings herein, many further embodiments, alternatives in design and uses of the embodiments of the instant invention will be apparent to those of skill in the art. As such, it is not intended that the invention be limited to the particular illustrative embodiments, alternatives, and uses described above but instead by the claims presented hereafter.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. A system for use in minimally invasive surgery for introducing a visualization device into a body cavity, comprising:
a cylindrical tube providing a percutaneous optical path for a visualization device disposed inside of the cylindrical tube, the cylindrical tube including a distal end inside of a patient's body while a proximate end remains outside of the patient's body during minimally invasive surgery; and
a distal tip adapted to be attached to the distal end of the cylindrical tube and having a plurality of articulating components that are configured of being in either an entry state or a visualization, wherein;
the entry state is characterized by the plurality of articulating components forming a rigid sharp tip geometry capable of puncturing through a body wall and into an anatomical cavity near or adjacent to a surgical site, and
the visualization state is characterized by the plurality of articulating components diverting away from being in the percutaneous optical path.

17. The system of claim 16, wherein at least some of the plurality of articulating components are significantly transparent for the visualization device to image, through the at least some of the articulating transparent components, tissue surrounding the distal tip during the entry state.

18. The system of claim 16, wherein at least a portion of the visualization device is configured to extend beyond the length of the distal tip and towards the surgical site during the visualization state.

19. The system of claim 16, wherein the plurality of articulating components of the distal tip are configured to rotate about a compliant flexure hinge.

20. The system of claim 19, wherein the distal tip additionally comprises an integrated lighting means oriented to illuminate the tissue in one or both of the entry state and the visualization state.

21. The system of claim 16, wherein the tube comprises auxiliary channels configured for one or more of: to introduce a liquid into the body cavity, to introduce a liquid used to clean the objective lens of the visualization device, and to introduce a gas into the body cavity for insufflation.

22. A method for visualization during minimally invasive surgery, the method comprising:
providing a cylindrical tube that is configured as a percutaneous optical path for a visualization device disposed inside of the cylindrical tube, the cylindrical tube including a distal end inside of a patient's body while a proximate end remains outside of the patient's body during minimally invasive surgery; and
further providing a distal tip adapted to be attached to the distal end of the cylindrical tube and having a plurality of articulating components that are configured of being in either an entry state or a visual, wherein;
the entry state is characterized by the plurality of articulating components forming a rigid sharp tip geometry capable of puncturing through a body wall and into an anatomical cavity near or adjacent to a surgical site, and
the visualization state is characterised by the plurality of articulating components diverting away from being in the percutaneous optical path.

23. The method of claim 22, wherein at least some of the plurality of articulating components are significantly transparent for the visualization device to image, through the at least some of the articulating transparent components, tissue surrounding the distal tip during the entry state.

24. The method of claim 23, additionally comprising:
configuring the articulating components to transition from the entry state to a visualization state once the distal tip is inside the body cavity; and
configuring the visualization device to change its position within the cylindrical tube upon reaching, or during the transition to, the visualization state.

25. The method of claim 23, additionally comprising:
positioning an actuating component on the proximate end of the cylindrical tube for actuating an integrated light contained in the distal tip during the minimally invasive surgery.

26. A method for visualization during minimally invasive surgery, the method comprising:
inserting a cylindrical tube that is configured as a percutaneous optical path for a visualization device disposed inside of the cylindrical tube, the cylindrical tube including a distal end inside of a patient's body while proximate end remains outside of the patterns body during minimally invasive surgery using a distal tip adapted to be attached to the distal end of the cylindrical tube and having a plurality of articulating components that are configured of being in either an entry state or a visualization, wherein;
the entry state is characterized by the plurality of articulating components forming a rigid sharp tip geometry capable of puncturing through a body wall and into an anatomical cavity near or adjacent to a surgical site, and
the visualization state is characterized by the plurality of articulating components diverting away from being in the percutaneous optical path;
changing the distal tip from the entry state to the visualization state after the distal tip reaches a point near or at the surgical site; and
viewing, during the minimally invasive surgery, the surgical site at least in part via the visualization device disposed inside of the cylindrical tube.

27. The method of claim 26, wherein at least some of the plurality of articulating components are significantly transparent for the visualization device to image, through the at least some of the articulating transparent components, tissue surrounding the distal tip during the entry state.

28. The method of claim 26, additionally comprising:
configuring the articulating components to transition from the entry state to a visualization state once the distal tip is inside the body cavity; and
configuring the visualization device to change its position within the cylindrical tube upon reaching, or during the transition to, the visualization state.

29. The method of claim 26, additionally comprising:
positioning an actuating component on the proximate end of the cylindrical tube for actuating an integrated light contained in the distal tip during the minimally invasive surgery.

30. The method of claim 26, additional comprising:
rinsing an objective lens of the visualization device using a liquid introduced through one or more of the auxiliary channels of the cylindrical tube.

31. The method of claim 26, additionally comprising:
introducing an insufflating gas into the body cavity through one or more auxiliary channels of the cylindrical tube.

32. The method of claim 26, wherein the steps of inserting and viewing are performed without having to replace or temporarily remove the imaging device arranged inside of the cylindrical tube.

33. The method of claim 32, additionally comprising:
illuminating the tissue and surgical site using a light on or fixed about the distal dip, and controlled from the proximate portion of the cylindrical tube, during both puncturing thru tissue and the minimally invasive surgery.

34. The method of claim 26, additionally comprising:
obtaining a wide-view of the surgical site by displacing the visualization device disposed in the cylindrical tube just past the distal tip during the visualization state.

35. The method of claim 28, additionally comprising:
displacing tissue around the distal tip by rotating at least some of the plurality of articulating components about a hinge as they reach the visualization state.
